(19) United States
(12) Patent Application Publication
(10) Pub. No.: US 2024/0398835 A1
(43) Pub. Date: Dec. 5, 2024

(54) METHODS FOR TREATING CONGENITAL DISORDERS OF GLYCOSYLATION

(71) Applicants: Justin Donnelly, San Francisco, CA (US); Ethan Oren Perlstein, Oakland, CA (US); Diana Tong Roetting, Charlotte, NC (US); Clement Chow, Salt Lake City, UT (US)

(72) Inventors: Justin Donnelly, San Francisco, CA (US); Ethan Oren Perlstein, Oakland, CA (US); Diana Tong Roetting, Charlotte, NC (US); Clement Chow, Salt Lake City, UT (US)

(21) Appl. No.: 18/651,458

(22) Filed: Apr. 30, 2024

Related U.S. Application Data
(60) Provisional application No. 63/499,428, filed on May 1, 2023.

(51) Int. Cl. A61K 31/58; A61K 31/165; A61K 31/277; A61K 31/4741; A61K 31/519

(52) U.S. Cl. CPC A61K 31/58; A61K 31/165; A61K 31/277; A61K 31/4741; A61K 31/519

(57) ABSTRACT

The present disclosure relates generally to compounds and pharmaceutical compositions for decreasing glycosylation and treating congenital disorders of glycosylation.

### CROSS-REFERENCE TO RELATED APPLICATIONS

This application claims the benefit of priority to U.S. Provisional Patent Application No. 63/499,428, filed May 1, 2023, the contents of which are incorporated herein by reference in their entirety.

### FIELD

Provided herein are compounds and pharmaceutical compositions for decreasing glycosylation and treating congenital disorders of glycosylation.

### BACKGROUND

Congenital disorders of glycosylation (CDG) include more than 130 inborn errors of metabolism that affect N-linked, O-linked protein and lipid-linked glycosylation. CDGs are typically classified as Types I (CDG-1) and II (CDG-II).

Guanosine diphosphate (GDP)-mannose pyrophosphorylase A congenital disorder of glycosylation (GMPPA-CDG) is a rare disease, one of the congenital disorders of glycosylation. GMPPA-CDG is caused by mutations in the GMPPA gene, which encodes for GDP-mannose pyrophosphorylase A. This is the regulatory subunit of the enzyme that is responsible for the production of GDP-mannose, a sugar molecule that plays a critical role in the glycosylation of proteins and lipids in the body. Mutations in GMPPA-CDG are hypothesized to result in excessive GDP-mannose production and hyperglycosylation. GMPPA-CDG is characterized by a wide range of symptoms including developmental delay, intellectual disability, seizures, muscle weakness, and hypotonia. Other common features include facial dysmorphism, liver dysfunction, and abnormal bleeding. There is currently no cure for GMPPA-CDG and treatment is mainly supportive and symptomatic, focusing on the management of individual symptoms as they arise.

### SUMMARY

The present disclosure, in one embodiment, provides a method for treating a condition or disorder mediated, at least in part, by GDP-mannose pyrophosphorylase A in a patient in need thereof, comprising administering a therapeutically effective amount of a compound having a structure of:

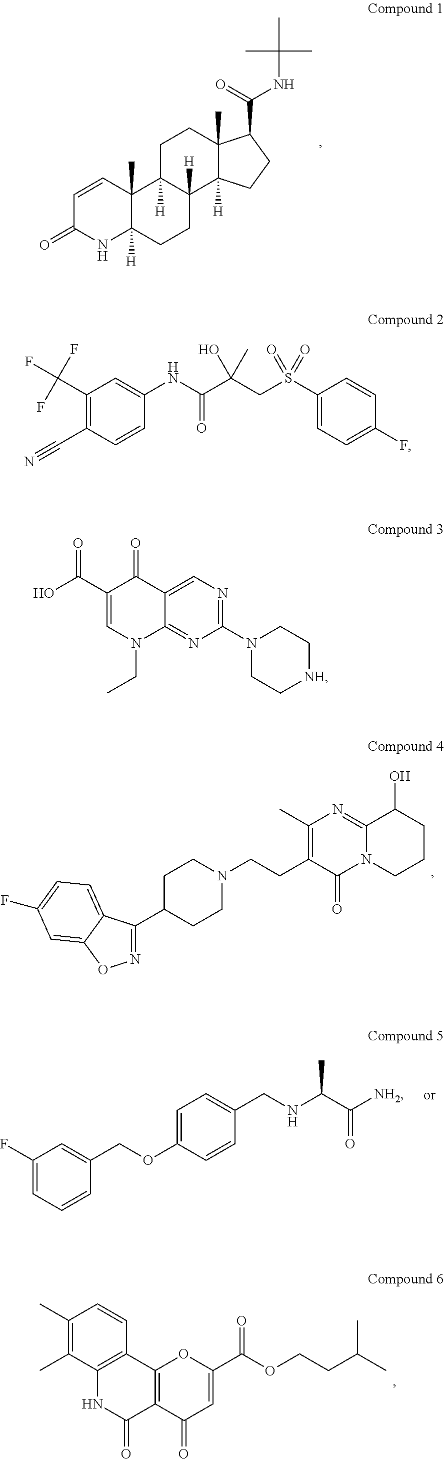

or a pharmaceutically acceptable salt of each thereof, or a combination thereof.

In some embodiments, the condition or disorder is GDP-mannose pyrophosphorylase A congenital disorder of glycosylation (GMPPA-CDG).

The present disclosure, in one embodiment, provides a method for treating a GMPPA deficiency in a patient in need thereof, comprising administering a therapeutically effective amount of a compound having a structure of Compound 1, Compound 2, Compound 3, Compound 4, Compound 5, or Compound 6, or a pharmaceutically acceptable salt of each thereof, or a combination thereof.

In some embodiments, the GMPPA deficiency is GMPPA-CDG.

The present disclosure, in one embodiment, provides a method for treating GMPPA-CDG in a patient in need thereof, comprising administering a therapeutically effective amount of a compound having a structure of Compound 1, Compound 2, Compound 3, Compound 4, Compound 5, or Compound 6, or a pharmaceutically acceptable salt of each thereof, or a combination thereof.

In some embodiments, the compound has a structure of Compound 1, Compound 2, Compound 3, or Compound 4, or a pharmaceutically acceptable salt of each thereof, or a combination thereof.

### BRIEF DESCRIPTION OF THE DRAWINGS

FIG. 1A illustrates GMPPA-knockout fly model without drugs administered, according to an embodiment. Larvae of GMPPA-knockout fly (highlighted with arrows) failed to emerge from the pupal case.

FIG. 1B illustrates GMPPA-knockout fly model with drugs administered, according to an embodiment. Larvae of GMPPA-knockout fly successfully emerged to adult flies.

FIG. 2 illustrates fly knockout model results for Compounds 1-6, according to an embodiment.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.05%. Also, to the term “about X” includes description of “X.”

Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Provided are also pharmaceutically acceptable salts, stereoisomers, mixture of stereoisomers, hydrates, solvates, solid forms, and tautomeric forms of the compounds described herein.

In many cases, the compounds of this disclosure are capable of forming acid and/or base salts by virtue of the presence of amino and/or carboxyl groups or groups similar thereto.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri (iso-propyl) amine, tri (n-propyl) amine, ethanolamine, 2-dimethylaminoethanol, piperazine, piperidine, morpholine, N-ethylpiperidine, and the like.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are non-superimposable mirror images of one another.

The term “solvate” refers to a complex formed by a combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” or “patient” refers to an animal, such as a mammal (including a human), that has been or will be the object of treatment, observation or experiment. The methods described herein may be useful in human therapy and/or veterinary applications. In some embodiments, the subject or patient is a mammal. In some embodiments, the subject or patient is a human.

The term “therapeutically effective amount” or “effective amount” of a compound described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a condition or disorder described herein, including but not limited to GMPPA deficiency. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Compounds, Pharmaceutical Compositions, and Modes of Administration

It is contemplated that the compounds provided herein are useful for treating a congenital disorder of glycosylation, such as GMPPA-CDG.

In some embodiments, the compound has the following structure:

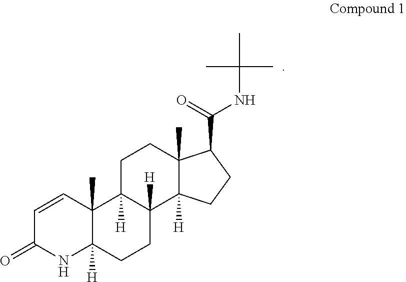

In some embodiments, the compound is a pharmaceutically acceptable salt of Compound 1. Compound 1, also known as finasteride, is commercially available and also may be synthesized according to methods known in the art.

In some embodiments, the compound has the following structure:

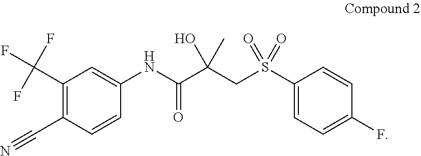 [SMILES: CC(O)(CS(=O)(=O)c1ccc(F)cc1)C(=O)Nc1ccc(C#N)c(C(F)(F)F)c1]

In some embodiments, the compound is a pharmaceutically acceptable salt of Compound 2. Compound 2, also known as N-(4-cyano-3-(trifluoromethyl)phenyl)-3-((4-fluorophenyl) sulfonyl)-2-hydroxy-2-methylpropanamide or bicalutamide, is commercially available and also may be synthesized according to methods known in the art.

In some embodiments, the compound has the following structure:

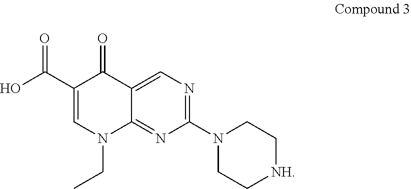 [SMILES: CCn1cc(C(=O)O)c(=O)c2cnc(N3CCNCC3)nc21]

In some embodiments, the compound is a pharmaceutically acceptable salt of Compound 3. Compound 3, also known as 8-Ethyl-5,8-dihydro-5-oxo-2-(1-piperazinyl)pyrido (2,3-d)pyrimidine-6-carboxylic acid or pipemidic acid, is commercially available and also may be synthesized according to methods known in the art.

In some embodiments, the compound has the following structure:

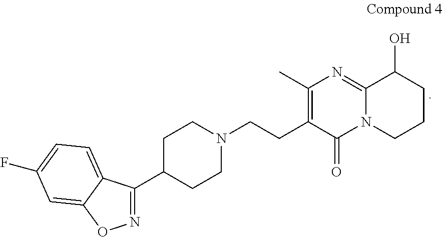 [SMILES: Cc1nc2n(c(=O)c1CCN1CCC(c3noc4cc(F)ccc34)CC1)CCCC2O]

In some embodiments, the compound is a pharmaceutically acceptable salt of Compound 4. Compound 4, also known as 9-hydroxyrisperidone, 3-(2-(4-(6-fluorobenzo[d]isoxazol-3-yl) piperidin-1-yl)ethyl)-9-hydroxy-2-methyl-6,7,8,9-tetrahydro-4H-pyrido[1,2-a]pyrimidin-4-one, or paliperidone, is commercially available and also may be synthesized according to methods known in the art.

In some embodiments, the compound has the following structure:

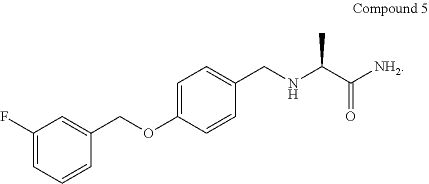 [SMILES: C[C@H](NCc1ccc(OCc2cccc(F)c2)cc1)C(N)=O]

In some embodiments, the compound is a pharmaceutically acceptable salt of Compound 5. Compound 5, also known as(S)-2-((4-((3-fluorobenzyl)oxy)benzyl)amino) propanamide or safinamide, is commercially available and also may be synthesized according to methods known in the art.

In some embodiments, the compound has the following structure:

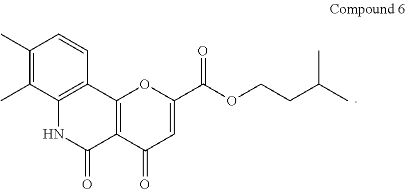 [SMILES: Cc1ccc2c([nH]c(=O)c3c(=O)cc(C(=O)OCCC(C)C)oc32)c1C]

In some embodiments, the compound is a pharmaceutically acceptable salt of Compound 6. Compound 6, also known as 3-methylbutyl 7,8-dimethyl-4,5-dioxo-6H-pyrano[3,2-c]quinoline-2-carboxylate or repirinast, is commercially available and also may be synthesized according to methods known in the art.

Also provided herein, in some embodiments, are pharmaceutical compositions that comprise one or more of the compounds described herein, or a pharmaceutically acceptable salt thereof, and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, such as orally or nasally, from devices that deliver the formulation in an appropriate manner.

### Kits

Provided herein are also kits that include a compound of the disclosure and suitable packaging. In one embodiment, a kit further includes instructions for use. In one aspect, a kit includes a compound of the disclosure and a label and/or instructions for use of the compounds in the treatment of the indications, including the diseases or conditions, described herein.

Provided herein are also articles of manufacture that include a compound described herein in a suitable container. The container may be a vial, jar, ampoule, preloaded syringe, and intravenous bag.

### Treatment Methods and Uses

A congenital disorder of glycosylation (CDG) is associated with deficient or defective glycosylation of various tissue proteins or lipids. Guanosine diphosphate (GDP)-mannose pyrophosphorylase A congenital disorder of glycosylation (GMPPA-CDG) is a rare disease with no cure. GMPPA is reported to serve as a regulatory subunit mediating feedback inhibition of glcosylation, and the defect in GMPPA leads to hyperglycosylation.

Provided herein are methods for treating decreasing glycosylation in a patient in need thereof comprising administering a therapeutically effective amount of a compound as described herein, or a combination of the compounds described herein, or a composition as described herein.

In some embodiments, decreasing glycosylation provides decreased levels of glycosylated glycoproteins, lysosomal enzymes, or serum proteins as compared to levels of glycoproteins, lysosomal enzymes, or serum proteins prior to administration. Measurement of glycosylated glycoproteins, lysosomal enzymes, or serum proteins are methods known in the art. See, e.g., Carchon et al., Clinical Chemistry, 50:1, 101-111 (2004).

Provided herein are methods for treating a condition or disorder mediated, at least in part, by GMPPA in a patient in need thereof comprising administering a therapeutically effective amount of a compound as described herein, or a combination of the compounds described herein, or a composition as described herein.

In some embodiments, the condition or disorder mediated, at least in part, by GMPPA, is a congenital disorder of glycosylation. In some embodiments, the congenital disorder of glycosylation is a Type I disorder (e.g. Ia, Ib, Ic, Id, Ie, If, Ih, Ii, Ij, Ik, IL, Im, In, Io, Ip, Iq, Ir, DPM2-CDG, TUSC3-CDG, GMPPA-CDG, MAGT1-CDG, DHDDS-CDG, and I/IIx). In some embodiments, the congenital disorder of glycosylation is a Type II disorder (e.g. Ila, Ilb, IIc, IId, Ile, IIf, Ilg, IIh, IIi, IIj, IIL, ATP6VOA2-CDG, MANIBI-CDG, and ST3GAL3-CDG).

Provided herein are methods for treating a congenital disorder of glycosylation in a patient in need thereof comprising administering a therapeutically effective amount of a compound as described herein, or a combination of the compounds described herein, or a composition as described herein. In some embodiments, a method for treating a congenital disorder of glycosylation in a patient in need thereof comprises administering a therapeutically effective amount of Compound 1 or a pharmaceutically acceptable salt thereof. In some embodiments, a method for treating a congenital disorder of glycosylation in a patient in need thereof comprises administering a therapeutically effective amount of Compound 2 or a pharmaceutically acceptable salt thereof. In some embodiments, a method for treating a congenital disorder of glycosylation in a patient in need thereof comprises administering a therapeutically effective amount of Compound 3 or a pharmaceutically acceptable salt thereof. In some embodiments, a method for treating a congenital disorder of glycosylation in a patient in need thereof comprises administering a therapeutically effective amount of Compound 4 or a pharmaceutically acceptable salt thereof. In some embodiments, a method for treating a congenital disorder of glycosylation in a patient in need thereof comprises administering a therapeutically effective amount of Compound 5 or a pharmaceutically acceptable salt thereof. In some embodiments, a method for treating a congenital disorder of glycosylation in a patient in need thereof comprises administering a therapeutically effective amount of Compound 6 or a pharmaceutically acceptable salt thereof.

In some embodiments, the congenital disorder of glycosylation is a Type I disorder. In some embodiments, the congenital disorder of glycosylation is a Type II disorder.

In some embodiments, the congenital disorder of glycosylation is GMPPA-CDG.

Provided herein are methods for treating a GMPPA deficiency in a patient in need thereof comprising administering a therapeutically effective amount of a compound as described herein, or a combination of the compounds described herein, or a composition as described herein.

Provided herein are methods for treating GMPPA-CDG in a patient in need thereof comprising administering a therapeutically effective amount of a compound as described herein, or a combination of the compounds described herein, or a composition as described herein.

It is contemplated herein that, in some embodiments, a compound described herein may be useful for treating congenital disorders of glycosylation (CDG). In some embodiments, a compound described herein may be useful for treating GMPPA-CDG.

In any of the embodiments described herein, a patient is administered one or more of the compounds described herein. The one or more compounds can be administered simultaneously or sequentially.

In any of the embodiments described herein, a patient is administered a pharmaceutical composition that comprises one or more of the compounds described herein.

In any of the embodiments described herein, the patient is further administered a therapeutically effective amount of another therapeutic agent useful for decreasing glycosylation (e.g., tunicamycin). The another therapeutic agent may be administered simultaneously or sequentially with a compound, or compounds, described herein or a composition described herein.

### Dosing

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present disclosure or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In some embodiments, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1-GMPPA Knockout Fly

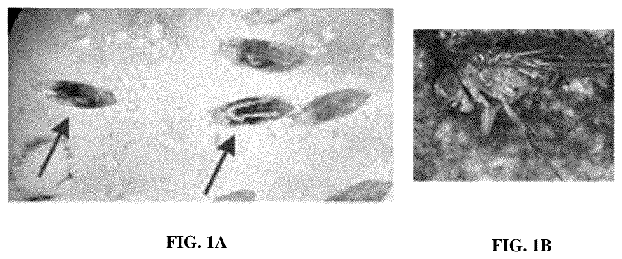

GMPPA-knockout Drosophila was prepared using RNAi under the control of a Tubulin-GAL4 promoter. It was confirmed that the GMPPA-knockout Drosophila larvae were 100% lethal and fail to emerge from the pupal case, as shown in FIG. 1A. Under normal rearing conditions, no single adult GMPPA-knockout fly was observed.

### Example 2-Fruit Fly Screening

Each of 1040 compounds from the Prestwick Chemical Library® members were provided at 10 mM in DMSO, then diluted to 1 mM in phosphate buffered saline (PBS). Then each 1 mM solution was added to a vial containing fly food, resulting in 200-fold dilution of the compound in fly food, to provide 5 μM concentration of each compound. DMSO was used as a negative control.

An average 19 GMPPA-knockout flies (pre-mated) were added to each vial, and were allowed to lay eggs on each drugged food and larvae were allowed to develop and pupate while feeding on drugged food, so they receive the compound for the duration of their lives. Each fly good/drug was monitored and scored for the development of adult flies.

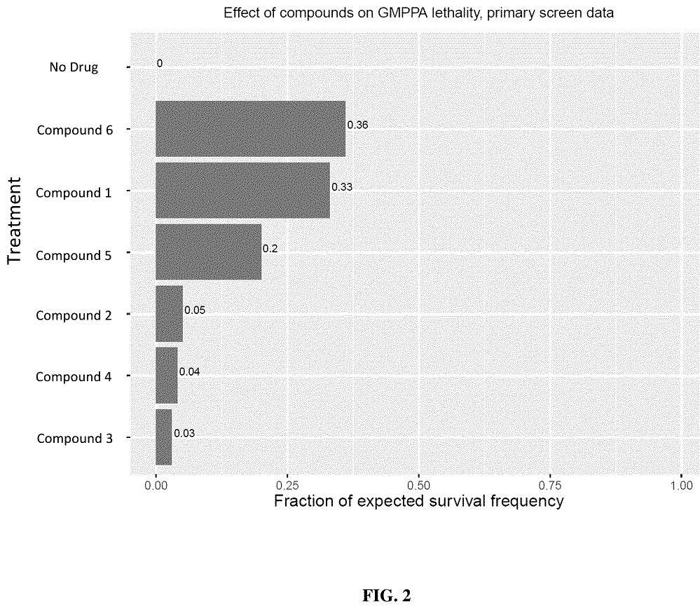

Drugs that resulted in development of adult flies were classified as “hits,” as the drug allowed the GMPPA knockout fly larvae to restore proper larval development and pupate, as shown in FIG. 1B. Fully developed adult flies in each vial were genotyped in order to quantify their Mendelian ratio. The negative control (DMSO, in the absence of drug) yielded no flies with the knockdown, while a theoretical complete rescue would be expected to produce a Mendelian ratio of 1:1 (knockdown vs control). The strength of each hit was determined by where the resulting Mendelian ratio fell on the spectrum between these two extremes. Six of the compounds (Compounds 1-6) showed some knockdown flies that survived to adulthood. The six hits (Compounds 1-6) and their strength is shown in FIG. 2.

It is contemplated that Compounds 1-6 restored control of glycosylation caused by the defect in GMPPA, namely hyperglycosylation, or reversed any other detrimental effects caused by the defect of GMPPA, such that larvae were allowed to develop and pupate. It is expected that Compounds 1-6 will reverse or alleviate symptoms of GMPPA-CDG, caused by defect in the orthologous human GMPPA gene.

### Example 3-C. elegans Model Assay

Five lines of GMPPA disorder models were generated in C. elegans using CRISPR/Cas9 gene editing:
One line GMPPA Human (Y47DA9.1 C. elegans) knockout (GMPPA KO)
Two lines T292P Human (S272P C. elegans) point mutation (T292P PM1, T292P PM2)
Two lines R318W Human (R298W C. elegans) point mutation (R318W PM1, R318W PM2)

These animals were tested for growth delays, temperature sensitivity, and mitotic stress sensitivity, with the dual goals of validating C. elegans as a model for studying GMPPA-CDG and identifying a robust phenotype that could be leveraged in future screening of novel pharmaceuticals for the treatment of the deleterious effects of this mutation.

All five lines of C.elegans model containing alterations of the GMPPA gene showed a large decrease in size at early larval stages. This phenotype became less pronounced as animals reached adulthood. Further, all five lines of C.elegans model containing alterations of the GMPPA gene also showed increased acute sensitivity to heat-induced stress, though the chronic response to heat shock was not different from control animals. These results indicate that the loss of the GMPPA gene in C. elegans has phenotypic consequences and that the T292P and R318W point mutations also cause a loss of GMPPA function.

To assess the effect of Compounds 1-6 on mitigating the growth deficit of C. elegans carrying a mutation in the GMPPA gene, the growth of the five mutant GMPPA C. elegans lines that were either untreated or treated with one of 6 different drugs (Compounds 1-6), at different concentrations, was measured. C. elegans were allowed to develop to the LI larval stage in the liquid solution. LI C. elegans were then placed onto plates seeded with inactivated OP50 containing the appropriate drug treatment in 0.1% DMSO, and were kept at 20° C. throughout the assay. About thirty 30 C. elegans synchronized at the LI stage of each genotype were placed onto 3 replicate plates containing the appropriate drug treatment and allowed to grow to adulthood at 20° C. Plates were imaged at the same time each day using the WormLab system (MBF Bioscience). Worm area was measured using the WormLab software. Animal sizes of each treatment were compared to sizes of untreated N2 control animals, as well as untreated animals of the same genotype on each day using ANOVA, followed by post-hoc t-tests corrected for multiple comparisons using the Benjamini-Hochberg correction procedure. Analyses were performed and plotted using custom R code.

To study the effects of the different drugs on growth, the size (body area) of animals treated with each concentration of each drug was compared to the size (body area) of untreated animals of the same genotype on the same day. The results are shown in Tables 1-6.

TABLE 1
Compound 1

| | | | % Increase | | |
|---|---|---|---|---|---|
| | | | size | | |
| Concen- | | | (compared to | | Adjusted |
| tration | Genotype | Stage | untreated) | P-Value | P-Value |
| 20 μM | R318W PM 1 | L3 | 10.73 | 0.0061 | 0.0208 |
| 2 μM | R318W PM 1 | Young Adult | 12.70 | 0.0010 | 0.0018 |
| | | Adult (day 5) | 9.08 | 0.0008 | 0.0012 |
| | GMPPA KO | Adult (day 4) | 6.47 | 0.0058 | 0.0155 |
| 200 nM | R318W PM 1 | L3 | 13.73 | 0.0007 | 0.0046 |
| | | Young Adult | 16.92 | 0.0002 | 0.0005 |
| | | Adult (day 5) | 7.28 | 0.0054 | 0.0065 |
| 20 nM | R318W PM 1 | L3 | 12.77 | 0.0008 | 0.0046 |
| | | Young Adult | 11.59 | 0.004 | 0.0069 |
| | | Adult (day 5) | 8.79 | 0.0008 | 0.0012 |

TABLE 2
Compound 2

| | | | % Increase | | |
|---|---|---|---|---|---|
| | | | size | | |
| Concen- | | | (compared to | | Adjusted |
| tration | Genotype | Stage | untreated) | P-Value | P-Value |
| 20 μM | R318W PM 1 | Adult (day 5) | 6.98 | 0.0058 | 0.0067 |
| 2 μM | R318W PM 1 | Young Adult | 15.14 | 0.0004 | 0.0009 |
| | | Adult (day 5) | 13.14 | <0.0001 | <0.0001 |
| | R318W PM 2 | Young Adult | 20.08 | 0.0006 | 0.0051 |
| | | Adult (day 5) | 9.82 | 0.0001 | 0.0014 |
| 200 μM | R318W PM 1 | Young Adult | 15.18 | 0.0001 | 0.0005 |
| | | Adult (day 5) | 13.42 | <0.0001 | 0.0001 |
| | GMPPA KO | Adult (day 4) | 10.83 | <0.0001 | 0.0001 |
| | T292P PM 1 | Young Adult | 16.12 | 0.0011 | 0.0138 |
| 20 μM | R318W PM 1 | Young Adult | 9.40 | 0.0247 | 0.0329 |
| | GMPPA KO | Adult (day 4) | 9.60 | <0.0001 | 0.0001 |
| | R318W PM 1 | Adult (day 5) | 12.37 | <0.0001 | <0.0001 |

TABLE 3
Compound 3

| | | | % Increase | | |
|---|---|---|---|---|---|
| | | | size | | |
| Concen- | | | (compared to | | Adjusted |
| tration | Genotype | Stage | untreated) | P-Value | P-Value |
| 20 μM | R318W PM 1 | L3 | 14.96 | 0.0003 | 0.0041 |
| | | Young Adult | 9.03 | 0.0217 | 0.0307 |
| | | Adult (day 5) | 12.83 | <0.0001 | <0.0001 |
| | R318W PM 2 | L3 | 11.65 | 0.0115 | 0.0346 |
| | | Young Adult | 14.52 | 0.0113 | 0.0451 |
| | | Adult (day 5) | 5.57 | 0.0068 | 0.0411 |
| 2 μM | R318W PM 1 | Young Adult | 18.05 | 0.0001 | 0.0005 |
| | | Adult (day 5) | 8.12 | 0.0031 | 0.0042 |
| | R318W PM 2 | Adult (day 4) | 11.20 | 0.0001 | 0.0008 |
| | GMPPA KO | Young Adult | 12.64 | 0.0042 | 0.0350 |
| | | Adult (day 4) | 12.37 | <0.0001 | <0.0001 |
| | T292P PM 1 | Adult (day 5) | 14.96 | 0.0002 | 0.0026 |
| 200 μM | R318W PM 1 | Young Adult | 14.43 | 0.0004 | 0.0009 |
| | | Adult (day 5) | 10.51 | 0.0002 | 0.0004 |
| | GMPPA KO | Adult (day 4) | 7.92 | 0.0002 | 0.0012 |
| | R318W PM 2 | Adult (day 4) | 8.02 | 0.0123 | 0.0491 |
| | | Adult (day 5) | 5.96 | 0.0111 | 0.0443 |
| 20 μM | R318W PM 1 | Young Adult | 15.84 | 0.0001 | 0.0005 |
| | | Adult (day 5) | 12.27 | <0.0001 | 0.0001 |

TABLE 4
Compound 4

| | | | % Increase | | |
|---|---|---|---|---|---|
| | | | size | | |
| Concen- | | | (compared to | | Adjusted |
| tration | Genotype | Stage | untreated) | P-Value | P-Value |
| 20 μM | R318W PM 1 | Adult (day 5) | 10.72 | 0.0001 | 0.0002 |
| | T292P PM 1 | Adult (day 5) | 6.70 | 0.0057 | 0.0459 |
| 2 μM | R318W PM 1 | Young Adult | 14.09 | 0.0004 | 0.0009 |
| | | Adult (day 5) | 9.47 | 0.0003 | 0.0006 |
| | GMPPA KO | Adult (day 4) | 6.86 | 0.0033 | 0.0114 |
| | R318W PM 2 | Adult (day 5) | 6.08 | 0.0033 | 0.0277 |
| 200 nM | R318W PM 2 | Adult (day 4) | 8.35 | 0.0098 | 0.0472 |
| | R318W PM 1 | Adult (day 5) | 11.03 | 0.0001 | 0.0001 |
| 20 nM | R318W PM 1 | Young Adult | 11.38 | 0.0052 | 0.0083 |
| | | Adult (day 5) | 10.32 | 0.0001 | 0.0002 |
| | GMPPA KO | Adult (day 4) | 7.18 | 0.0023 | 0.0093 |

TABLE 5
Compound 5

| | | | % Increase | | |
|---|---|---|---|---|---|
| | | | size | | |
| Concen- | | | (compared to | | Adjusted |
| tration | Genotype | Stage | untreated) | P-Value | P-Value |
| 2 μM | R318W PM 1 | L3 | 16.41 | 0.0001 | 0.0023 |
| | | Young Adult | 16.21 | 0.0002 | 0.0005 |
| | | Adult (day 5) | 9.47 | 0.0008 | 0.0012 |
| | R318W PM 2 | Adult (day 5) | 5.51 | 0.0093 | 0.0443 |
| 200 nM | R318W PM 1 | Adult (day 5) | 9.35 | 0.0034 | 0.0043 |
| 20 nM | R318W PM 1 | Adult (day 5) | 11.09 | 0.0001 | 0.0002 |

TABLE 6
Compound 6

| | | | % Increase | | |
|---|---|---|---|---|---|
| | | | size | | |
| Concen- | | | (compared to | | Adjusted |
| tration | Genotype | Stage | untreated) | P-Value | P-Value |
| 20 μM | R318W PM 1 | L3 | 13.93 | 0.0031 | 0.0124 |
| | | Young Adult | 18.41 | 0.0001 | 0.0005 |
| | | Adult (day 5) | 10.80 | 0.0009 | 0.0012 |
| | GMPPA KO | Young Adult | 13.22 | 0.0044 | 0.0350 |
| | R318W PM 2 | Young Adult | 22.20 | 0.0010 | 0.0063 |
| 2 μM | R318W PM 1 | Young Adult | 16.84 | 0.0001 | 0.0005 |
| | | Adult (day 5) | 7.41 | 0.0075 | 0.0082 |
| | GMPPA KO | Adult (day 4) | 6.58 | 0.0054 | 0.0155 |
| | R318W PM 2 | Young Adult | 18.25 | 0.0021 | 0.0099 |
| | | Adult (day 4) | 8.97 | 0.0026 | 0.0210 |
| 200 nM | R318W PM 1 | Young Adult | 18.38 | <0.0001 | 0.0002 |
| | | Adult (day 5) | 12.15 | <0.0001 | <0.0001 |
| | R318W PM 2 | Young Adult | 25.23 | <0.0001 | 0.0001 |
| | | Adult (day 4) | 7.96 | 0.0036 | 0.0216 |
| | T292P PM 2 | Young Adult | 13.63 | 0.0013 | 0.0157 |
| 20 nM | R318W PM 1 | L3 | 18.59 | 0.0013 | 0.0063 |
| | | Young Adult | 27.26 | <0.0001 | <0.0001 |
| | | Adult (day 5) | 11.15 | 0.0001 | 0.0002 |
| | R318W PM 2 | Young Adult | 20.62 | 0.0003 | 0.0035 |

As shown in Tables 1-6, all six compounds were effective at ameliorating the growth deficit produced by mutation in the GMPPA gene.

### Example 4-Fibroblast Assay

Compounds 1-6 are validated in a panel of GMPPA-CDG patient-derived fibroblast and healthy control cell lines by targeted metabolomics. Three (3) GMPPA-CDG patient derived fibroblasts and three (3) healthy age-matched control lines are cultured and each treated at one (1) fixed concentration (10 μM) of Compounds 1-6. Three (3) untreated (DMSO) patient and three (3) control lines are cultured as well. The fibroblast samples are analyzed and a custom panel of metabolites are quantified. The samples are further subject to glycoproteomics analysis.

### Certain Embodiments

Embodiment 1. A method for decreasing glycosylation in a patient in need thereof comprising administering a therapeutically effective amount of a compound having a structure of:

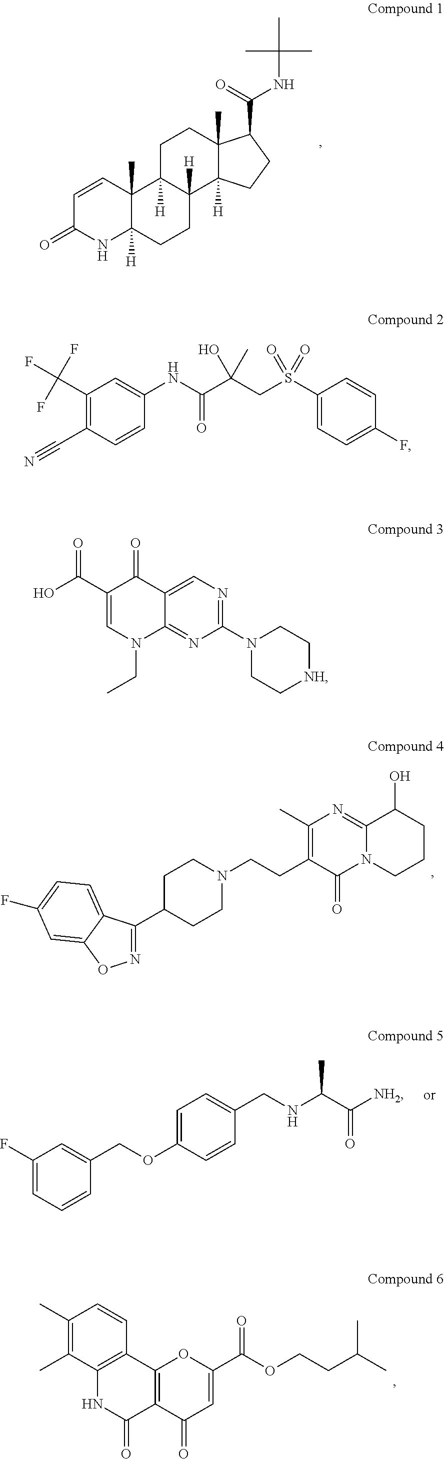

or a pharmaceutically acceptable salt of each thereof, or a combination thereof.

Embodiment 2. A method for decreasing glycosylation in a patient in need thereof comprising administering a pharmaceutical composition comprising a pharmaceutically acceptable carrier and a therapeutically effective amount of a compound having a structure of:

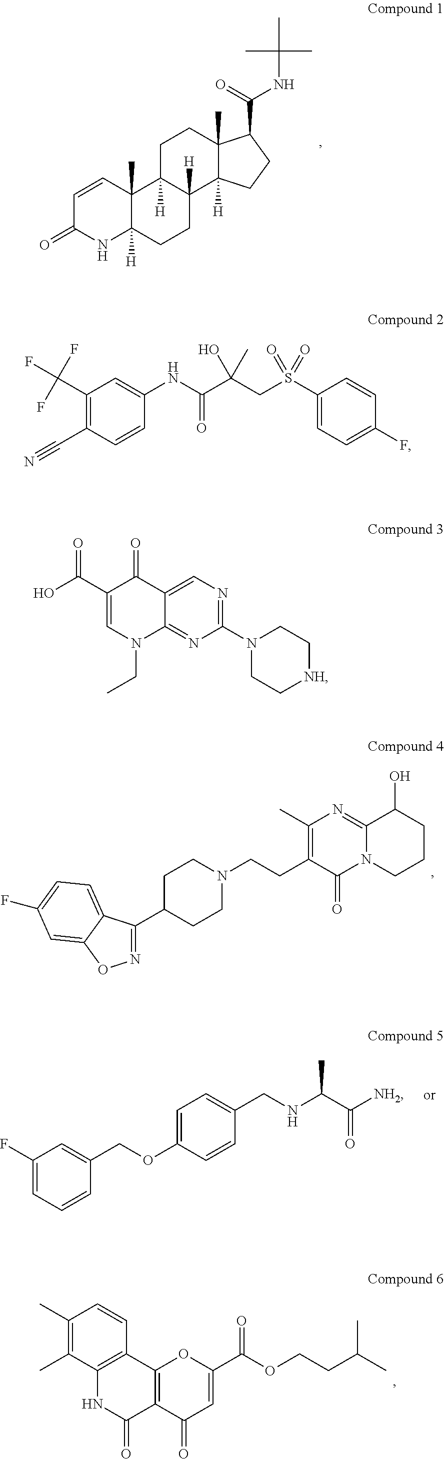

or a pharmaceutically acceptable salt of each thereof, or a combination thereof.

Embodiment 3. A method for treating a condition or disorder mediated, at least in part, by GDP-mannose pyrophosphorylase A in a patient in need thereof, comprising administering a therapeutically effective amount of a compound having a structure of:

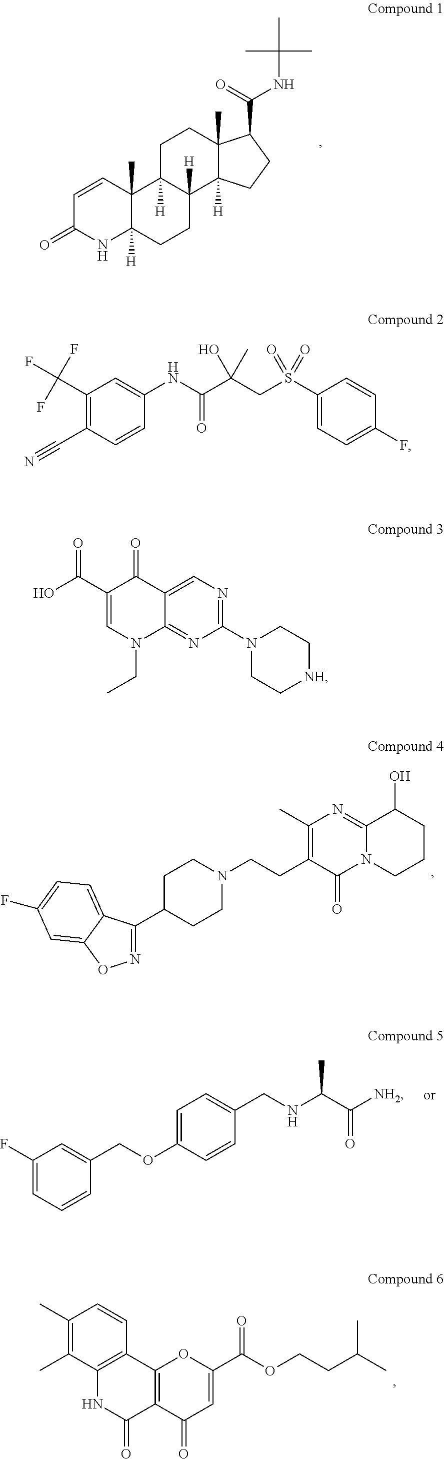

or a pharmaceutically acceptable salt of each thereof, or a combination thereof.

Embodiment 4. The method of Embodiment 3, wherein the condition or disorder is GDP-mannose pyrophosphorylase A congenital disorder of glycosylation (GMPPA-CDG).

Embodiment 5. A method for treating a condition or disorder mediated, at least in part, by GDP-mannose pyrophosphorylase A in a patient in need thereof, comprising administering a pharmaceutical composition comprising a pharmaceutically acceptable carrier and a therapeutically effective amount of a compound having a structure of:

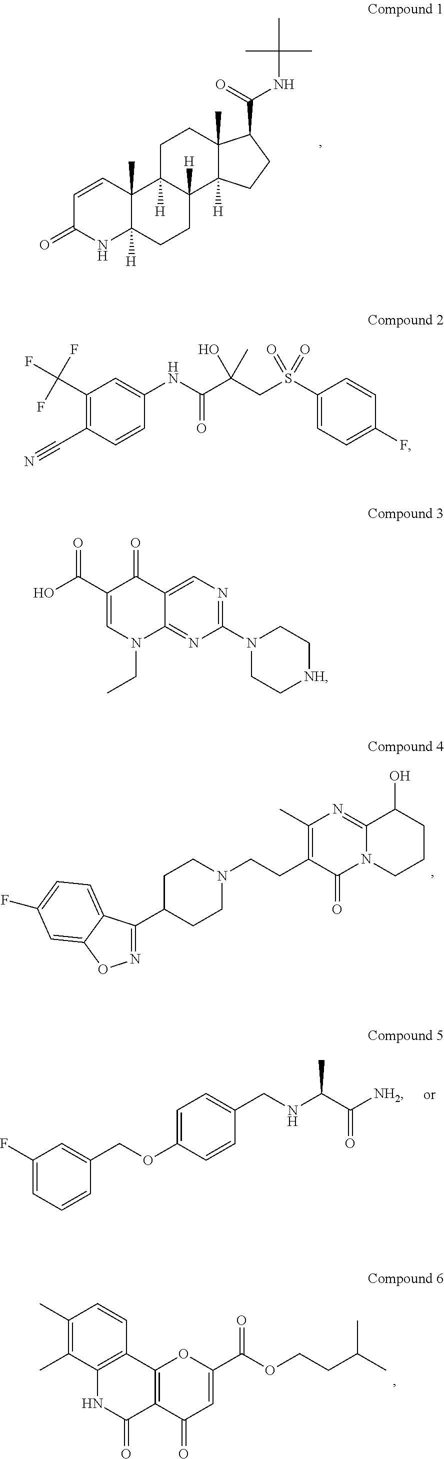

or a pharmaceutically acceptable salt of each thereof, or a combination thereof.

Embodiment 6. The method of Embodiment 5, wherein the condition or disorder is GMPPA-CDG.

Embodiment 7. A method for treating a congenital disorder of glycosylation in a patient in need thereof comprising administering a therapeutically effective amount of a compound having a structure of:

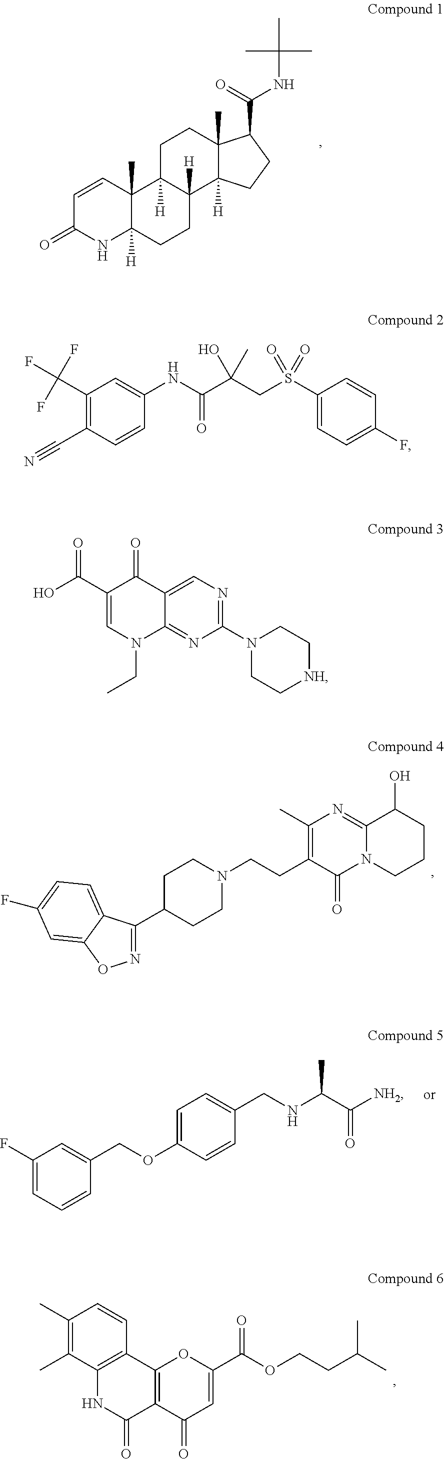

or a pharmaceutically acceptable salt of each thereof, or a combination thereof.

Embodiment 8. The method of Embodiment 7, wherein the congenital disorder of glycosylation is GMPPA-CDG.

Embodiment 9. A method for treating a congenital disorder of glycosylation in a patient in need thereof comprising administering a pharmaceutical composition comprising a pharmaceutically acceptable carrier and a therapeutically effective amount of a compound having a structure of:

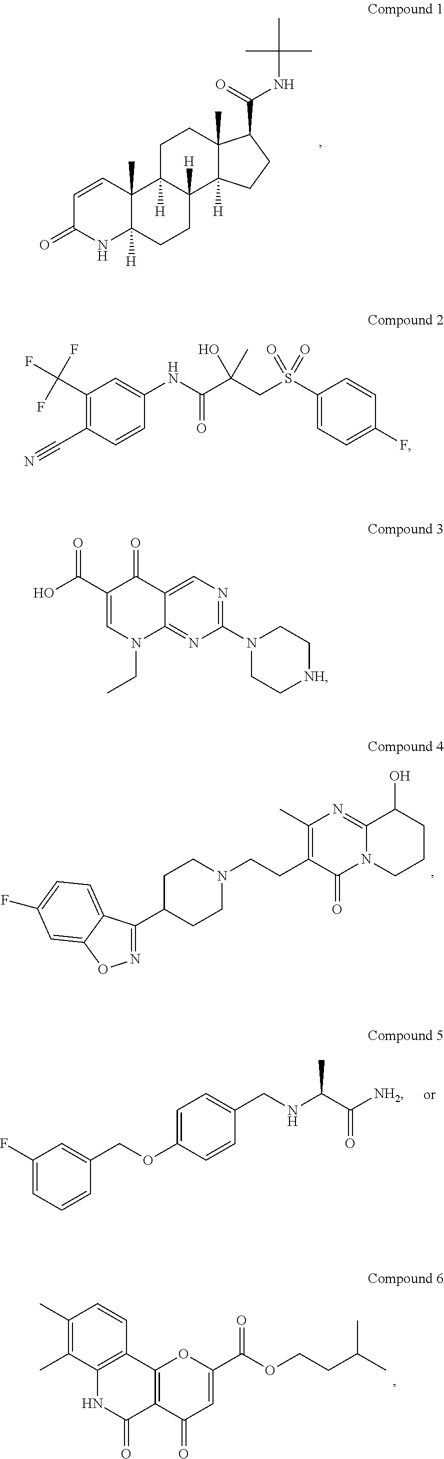

or a pharmaceutically acceptable salt of each thereof, or a combination thereof.

Embodiment 10. The method of Embodiment 9, wherein the congenital disorder of glycosylation is GMPPA-CDG.

Embodiment 11. A method for treating a GMPPA deficiency in a patient in need thereof comprising administering a therapeutically effective amount of a compound having a structure of:

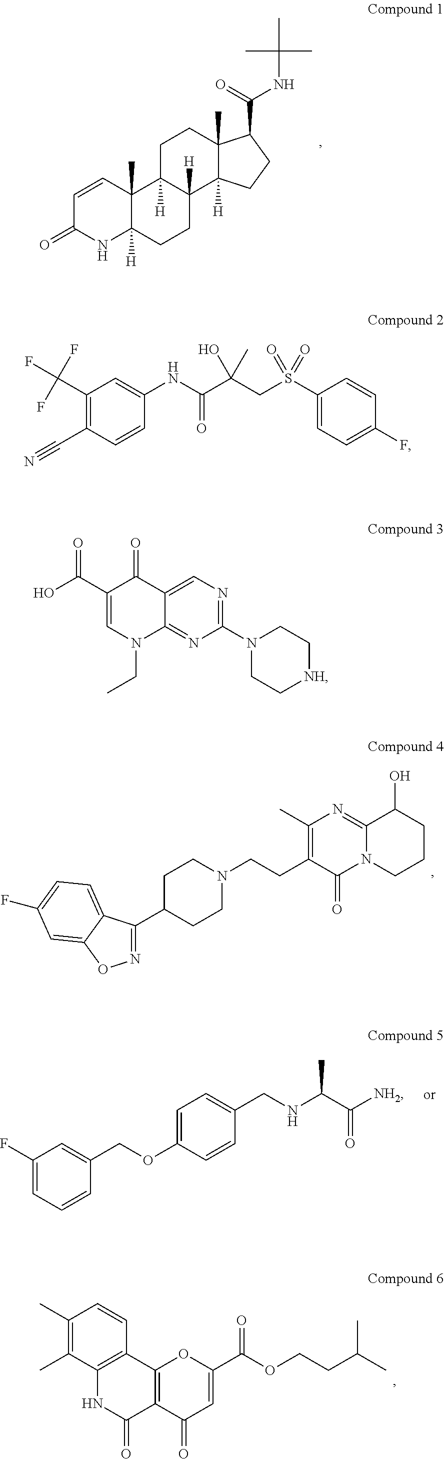

or a pharmaceutically acceptable salt of each thereof, or a combination thereof.

Embodiment 12. A method for treating a GMPPA deficiency in a patient in need thereof comprising administering a pharmaceutical composition comprising a pharmaceutically acceptable carrier and a therapeutically effective amount of a compound having a structure of:

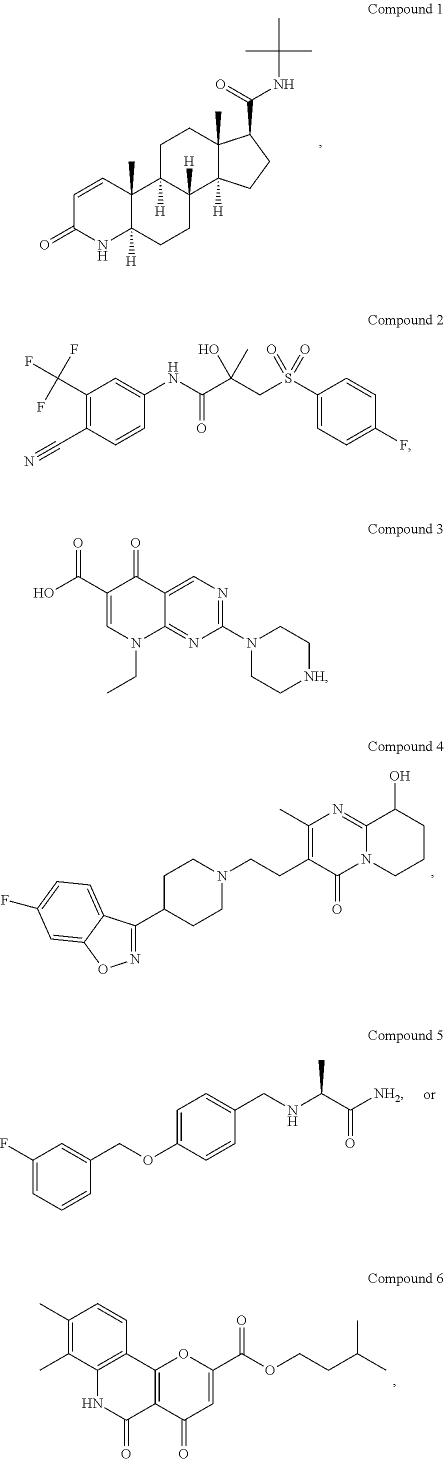

or a combination thereof.

Embodiment 13. A method for treating a congenital disorder of glycosylation in a patient in need thereof comprising administering a therapeutically effective amount of a compound having the structure of:

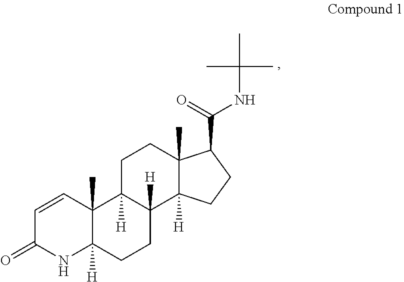

or a pharmaceutically acceptable salt thereof.

Embodiment 14. The method of Embodiment 13, wherein the congenital disorder of glycosylation is GMPPA-CDG.

Embodiment 15. A method for treating a congenital disorder of glycosylation in a patient in need thereof comprising administering a therapeutically effective amount of a compound having the structure of:

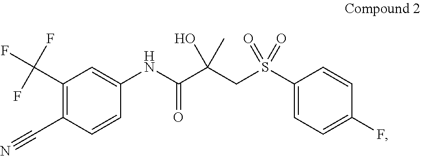 [SMILES: CC(O)(CS(=O)(=O)c1ccc(F)cc1)C(=O)Nc1ccc(C#N)c(C(F)(F)F)c1]

or a pharmaceutically acceptable salt thereof.

Embodiment 16. The method of Embodiment 15, wherein the congenital disorder of glycosylation is GMPPA-CDG.

Embodiment 17. A method for treating a congenital disorder of glycosylation in a patient in need thereof comprising administering a therapeutically effective amount of a compound having the structure of:

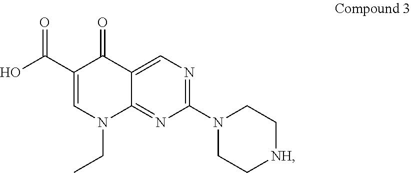 [SMILES: CCn1cc(C(=O)O)c(=O)c2cnc(N3CCNCC3)nc21]

or a pharmaceutically acceptable salt thereof.

Embodiment 18. The method of Embodiment 17, wherein the congenital disorder of glycosylation is GMPPA-CDG.

Embodiment 19. A method for treating a congenital disorder of glycosylation in a patient in need thereof comprising administering a therapeutically effective amount of a compound having the structure of:

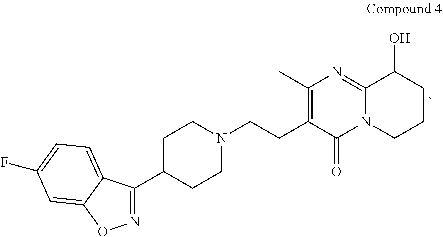 [SMILES: Cc1nc2n(c(=O)c1CCN1CCC(c3noc4cc(F)ccc34)CC1)CCCC2O]

or a pharmaceutically acceptable salt thereof.

Embodiment 20. The method of Embodiment 19, wherein the congenital disorder of glycosylation is GMPPA-CDG.

Embodiment 21. A method for treating a congenital disorder of glycosylation in a patient in need thereof comprising administering a therapeutically effective amount of a compound having the structure of:

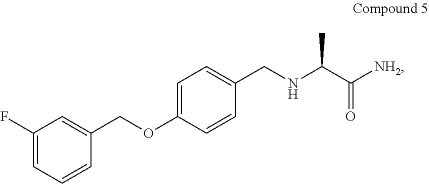 [SMILES: C[C@H](NCc1ccc(OCc2cccc(F)c2)cc1)C(N)=O]

or a pharmaceutically acceptable salt thereof.

Embodiment 22. The method of Embodiment 21, wherein the congenital disorder of glycosylation is GMPPA-CDG.

Embodiment 23. A method for treating a congenital disorder of glycosylation in a patient in need thereof comprising administering a therapeutically effective amount of a compound having the structure of:

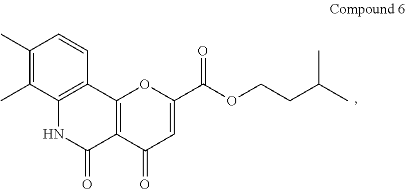 [SMILES: Cc1ccc2c([nH]c(=O)c3c(=O)cc(C(=O)OCCC(C)C)oc32)c1C]

or a pharmaceutically acceptable salt thereof.

Embodiment 24. The method of Embodiment 23, wherein the congenital disorder of glycosylation is GMPPA-CDG.

Embodiment 25. A method for treating GMPPA-CDG in a patient in need thereof comprising administering a therapeutically effective amount of a compound having a structure of:

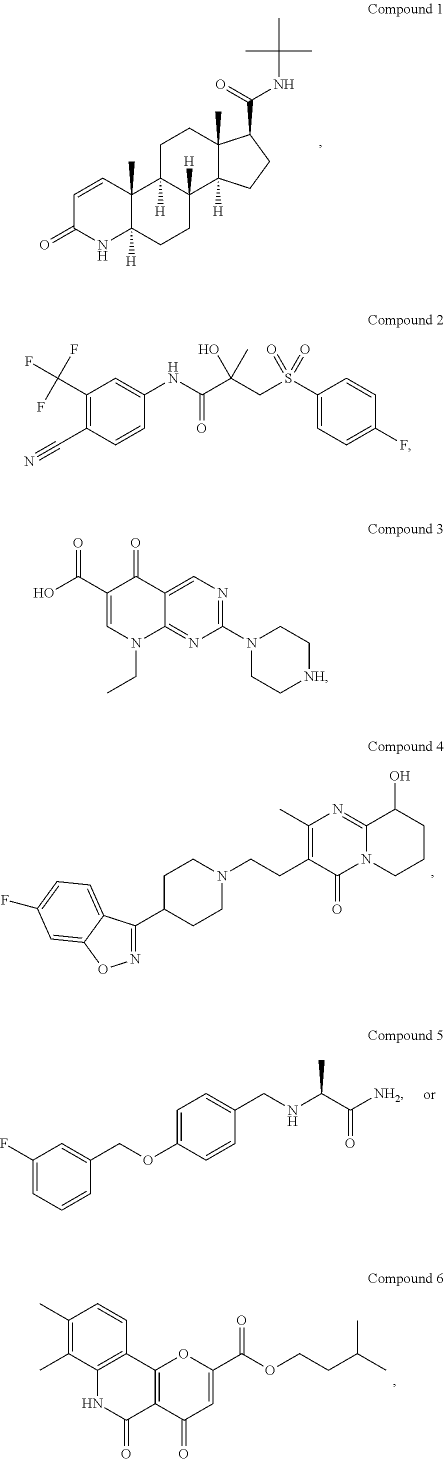

or a pharmaceutically acceptable salt of each thereof, or a combination thereof.

Embodiment 26. A method for treating GMPPA-CDG in a patient in need thereof comprising administering a pharmaceutical composition comprising a pharmaceutically acceptable carrier and a therapeutically effective amount of a compound having a structure of:

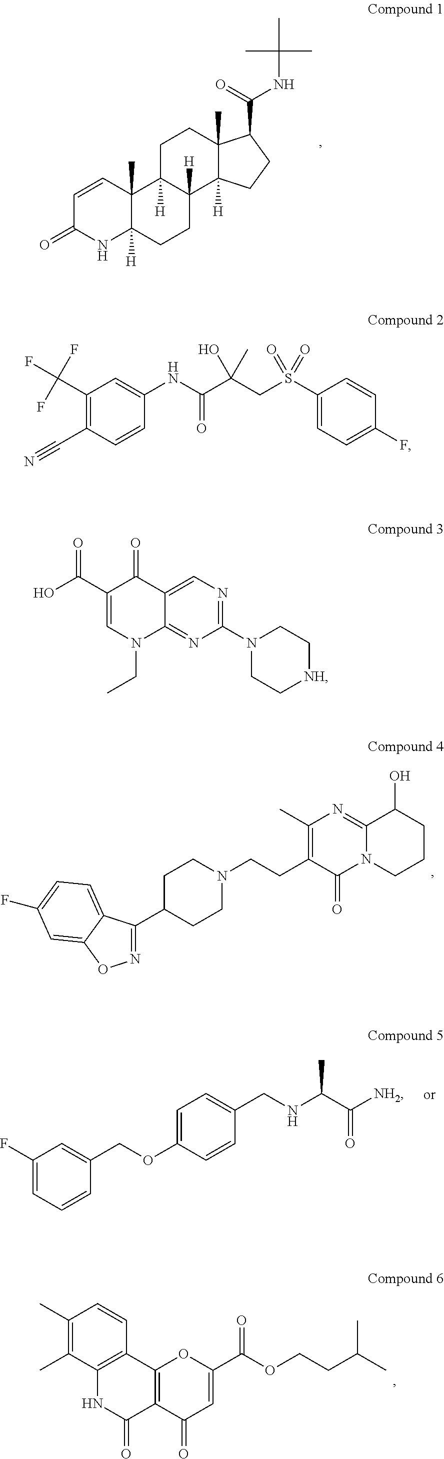

or a pharmaceutically acceptable salt of each thereof, or a combination thereof.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1-20. (canceled)

21. A method for treating a condition or disorder mediated, at least in part, by GDP-mannose pyrophosphorylase A in a patient in need thereof, comprising administering a therapeutically effective amount of a compound having a structure of: 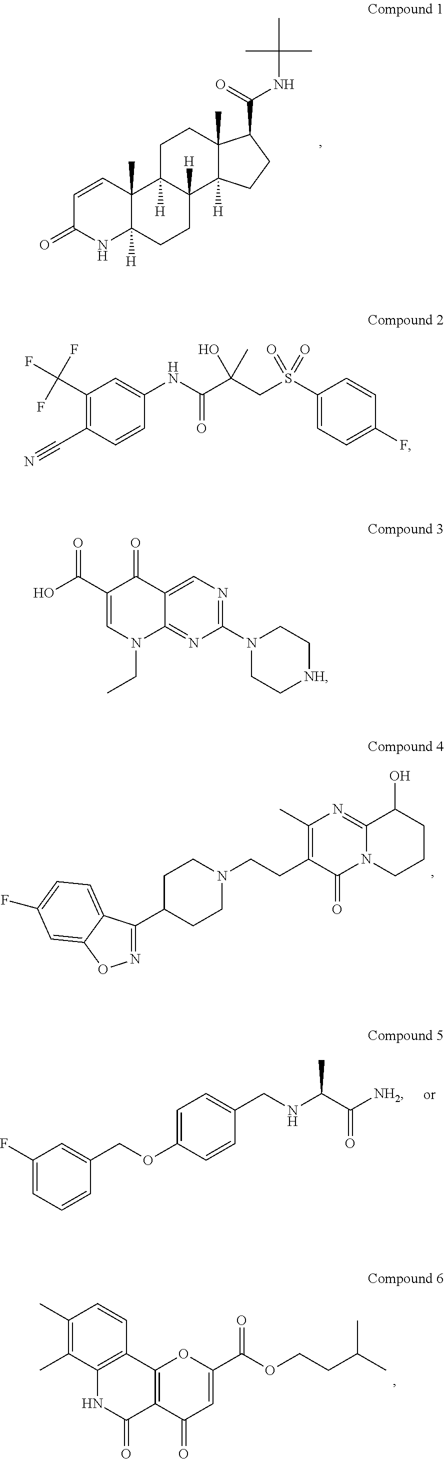
or a pharmaceutically acceptable salt of each thereof, or a combination thereof.

22. The method of claim 21, wherein the condition or disorder is GDP-mannose pyrophosphorylase A congenital disorder of glycosylation (GMPPA-CDG).

23. The method of claim 21, wherein the compound is Compound 1.

24. The method of claim 21, wherein the compound is Compound 2.

25. The method of claim 21, wherein the compound is Compound 3.

26. The method of claim 21, wherein the compound is Compound 4.

27. The method of claim 21, wherein the compound is Compound 5.

28. The method of claim 21, wherein the compound is Compound 6.

29. A method for treating a GMPPA deficiency in a patient in need thereof, comprising administering a therapeutically effective amount of a compound having a structure of: 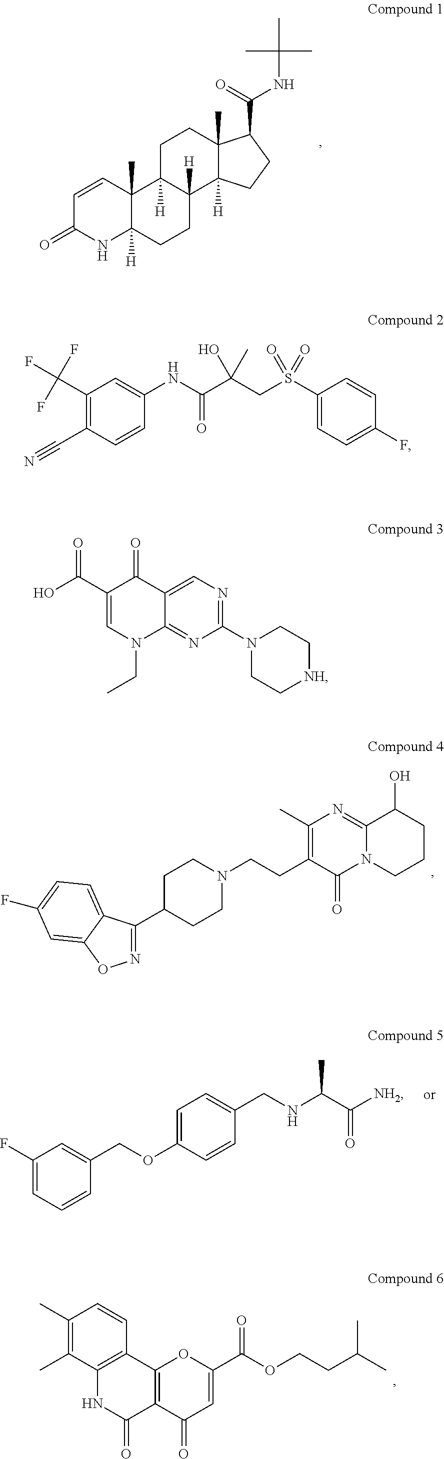
or a pharmaceutically acceptable salt of each thereof, or a combination thereof.

30. A method for treating GMPPA-CDG in a patient in need thereof comprising administering a therapeutically effective amount of a compound having a structure of: 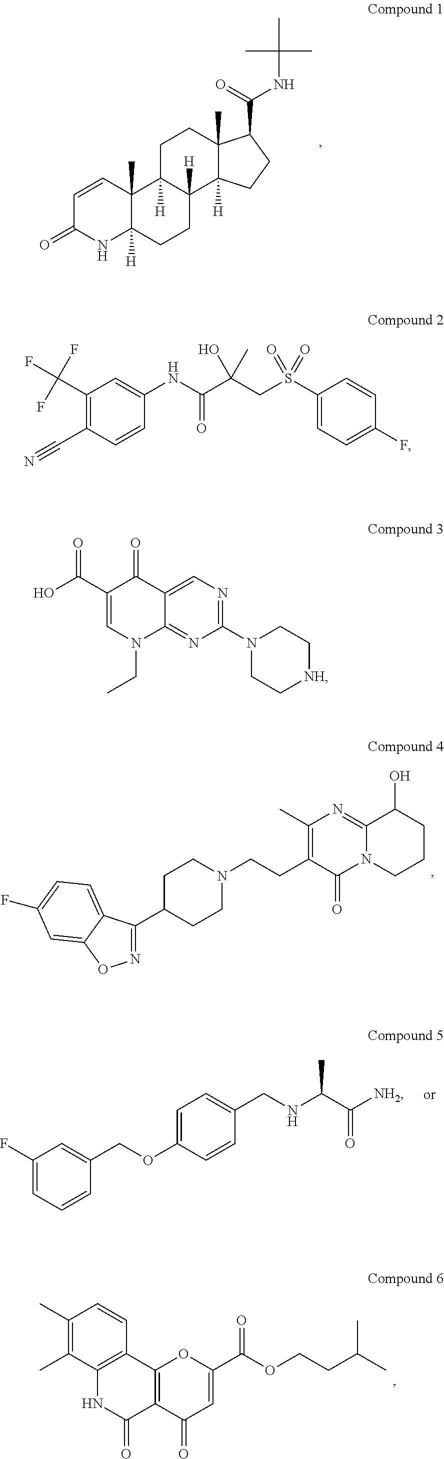
or a pharmaceutically acceptable salt of each thereof, or a combination thereof.

31. The method of claim 30, wherein the compound has a structure of: 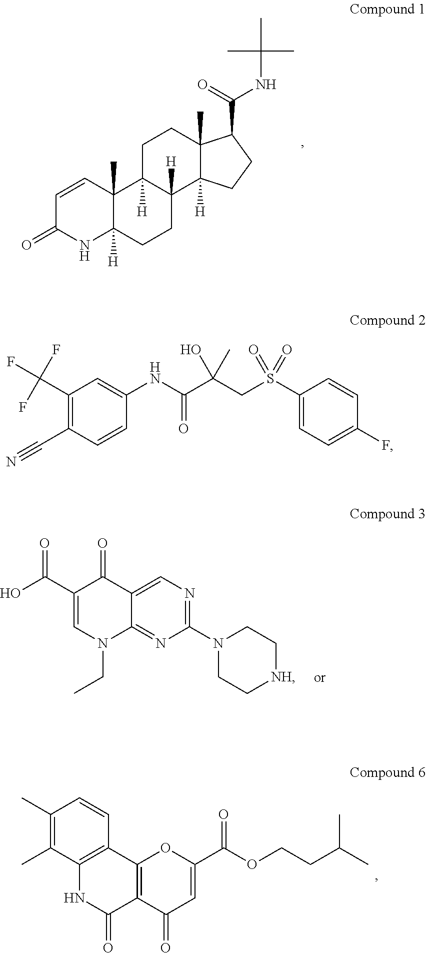
or a pharmaceutically acceptable salt of each thereof, or a combination thereof.

32. The method of claim 30, wherein the compound is Compound 1.

33. The method of claim 30, wherein the compound is Compound 2.

34. The method of claim 30, wherein the compound is Compound 3.

35. The method of claim 30, wherein the compound is Compound 4.

36. The method of claim 30, wherein the compound is Compound 5.

37. The method of claim 30, wherein the compound is Compound 6.
